(19) United States
(12) Patent Application Publication
(10) Pub. No.: US 2021/0346666 A1
(43) Pub. Date: Nov. 11, 2021

(54) INTRAVENOUS PORT CAP

(71) Applicant: Robin Erickson, Chetek, WI (US)

(72) Inventor: Robin Erickson, Chetek, WI (US)

(21) Appl. No.: 16/869,900

(22) Filed: May 8, 2020

(51) Int. Cl. A61M 39/02

(52) U.S. Cl. CPC A61M 39/0247; A61M 2039/0205; A61M 2039/0288

(57) ABSTRACT

An illustrated view of an exemplary port cap for application to a port allowing access to a blood stream is presented. The port cap is useful for eliminating adjustments to the port cap while being transported for chemotherapy, a blood draw and the like. The port cap is useful for sticking or adhering to the port. The port cap is further useful for providing an efficient and effective method for providing a numbing agent, such as lidocaine, to the port to prevent sensitivity and to eliminate the need to constantly adjust the port cap while for instance during moving or dressing/undressing.

### FIELD OF THE INVENTION

This invention relates to access to a blood stream. More particularly, it relates to application of anesthesia when accessing the blood stream.

### BACKGROUND

Blood is a body fluid in humans and other animals that delivers necessary substances such as nutrients and oxygen to the cells and transports metabolic waste products away from those same cells.

In vertebrates, it is composed of blood cells suspended in blood plasma. Plasma, which constitutes 55% of blood fluid, is mostly water (92% by volume), and contains proteins, glucose, mineral ions, hormones, carbon dioxide (plasma being the main medium for excretory product transportation), and blood cells themselves. Albumin is the main protein in plasma, and it functions to regulate the colloidal osmotic pressure of blood. The blood cells are mainly red blood cells (also called RBCs or erythrocytes), white blood cells (also called WBCs or leukocytes) and platelets (also called thrombocytes). The most abundant cells in vertebrate blood are red blood cells. These contain hemoglobin, an iron-containing protein, which facilitates oxygen transport by reversibly binding to this respiratory gas and greatly increasing its solubility in blood. In contrast, carbon dioxide is mostly transported extracellularly as bicarbonate ion transported in plasma.

Vertebrate blood is bright red when its hemoglobin is oxygenated and dark red when it is deoxygenated. Some animals, such as crustaceans and mollusks, use hemocyanin to carry oxygen, instead of hemoglobin. Insects and some mollusks use a fluid called hemolymph instead of blood, the difference being that hemolymph is not contained in a closed circulatory system. In most insects, this “blood” does not contain oxygen-carrying molecules such as hemoglobin because their bodies are small enough for their tracheal system to suffice for supplying oxygen.

Jawed vertebrates have an adaptive immune system, based largely on white blood cells. White blood cells help to resist infections and parasites. Platelets are important in the clotting of blood. Arthropods, using hemolymph, have hemocytes as part of their immune system.

Blood is circulated around the body through blood vessels by the pumping action of the heart. In animals with lungs, arterial blood carries oxygen from inhaled air to the tissues of the body, and venous blood carries carbon dioxide, a waste product of metabolism produced by cells, from the tissues to the lungs to be exhaled.

Medical terms related to blood often begin with hemo- or hemato- (also spelled haemo- and haemato-) from the Greek word αĩμα (haima) for “blood”. In terms of anatomy and histology, blood is considered a specialized form of connective tissue, given its origin in the bones and the presence of potential molecular fibers in the form of fibrinogen.

Chemotherapy (often abbreviated to chemo and sometimes CTX or CTx) is a type of cancer treatment that uses one or more anti-cancer drugs (chemotherapeutic agents) as part of a standardized chemotherapy regimen. Chemotherapy may be given with a curative intent (which almost always involves combinations of drugs), or it may aim to prolong life or to reduce symptoms (palliative chemotherapy). Chemotherapy is one of the major categories of the medical discipline specifically devoted to pharmacotherapy for cancer, which is called medical oncology.

The term chemotherapy has come to connote non-specific usage of intracellular poisons to inhibit mitosis, cell division. The connotation excludes more selective agents that block extracellular signals (signal transduction). The development of therapies with specific molecular or genetic targets, which inhibit growth-promoting signals from classic endocrine hormones (primarily estrogens for breast cancer and androgens for prostate cancer) are now called hormonal therapies. By contrast, other inhibitions of growth-signals like those associated with receptor tyrosine kinases are referred to as targeted therapy.

Often times when accessing the blood stream, whether for a blood draw or chemotherapy among other reasons for accessing the blood stream, a patient experiences pain or issues when a needle is inserted through skin layers to access a vein. When the patient has multiple reasons, such in the case of chemotherapy, the insertion of through the skin into the vein can cause sensitivities to the access areas. Although lidocaine is currently used to numb the area, the lidocaine is applied to the port and is kept in place by saran wrap or the like, adjustments by the patient are needed because of clothing, seat belts and constant movements while being transported to a hospital, clinic, etc. in order to keep the lidocaine over the access area (also referred to as a port).

Accordingly, in light of the foregoing, there is a need for an improved port cap to eliminate the constant adjusting in hours before chemotherapy or a blood draw. There is also a need to provide an efficient and effective longer-term numbing agent to the area to reduce the sensitivity at the port.

### BRIEF DESCRIPTION OF THE DRAWINGS

FIG. 1A is an illustrated view of an exemplary port cap.

FIG. 1B is an illustrated side view of the exemplary port cap shown in FIG. 1A.

FIG. 2 is an illustrated view of a use of the port cap shown in FIG. 1A.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

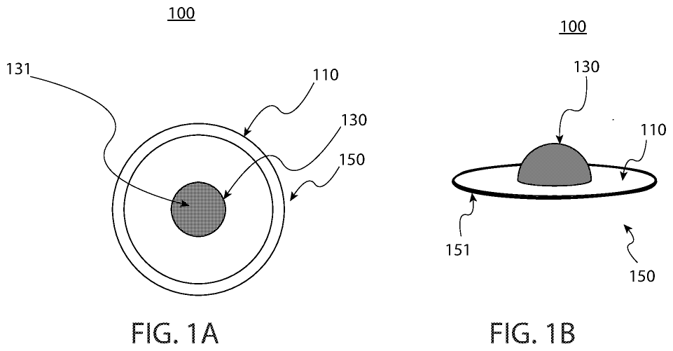

Referring to FIG. 1A and FIG. 1B, an illustrated view of an exemplary port cap 100 for application to a port allowing access to a blood stream is presented. The port cap 100 is useful for eliminating adjustments to the port cap 100 while being transported for chemotherapy, a blood draw and the like. The port cap 100 is useful for sticking or adhering to the port. The port cap 100 is further useful for providing an efficient and effective method for providing a numbing agent, such as lidocaine, to the port to prevent sensitivity and to eliminate the need to constantly adjust the port cap 100 while moving or dressing/undressing.

The port cap 100 is preferably circular in shape, however other shapes are hereby contemplated, including, but not limited to, square, rectangular, oval, etc. The port cap 100 is preferably made of a flexible material, such as latex, however other types of materials are hereby contemplated, including, but not limited to, plastic, nylon, etc. The port cap 100 is preferably clear in color, however other colors are hereby contemplated, including, but not limited to, translucent colors.

The port cap 100 has a top 110, a middle portion 130, and a bottom 150. The bottom 150, shown in FIG. 1B, has an adhesive 151 applied. The adhesive compound material is preferably made of a silicone material, however other types of materials are hereby contemplated, including, but not limited to, polyurethanes, acrylics, cyanocrylates, etc.

The middle portion 130 of the port cap 100 is a reservoir. A numbing agent 131 such as lidocaine, benzocaine, pramoxine, dibucaine, tetracaine and the like are inserted into the middle portion 130 of the port cap 100 to be applied to an application area or port of a patient. The numbing agent 131 is distributed through the bottom 150 of the port cap 100.

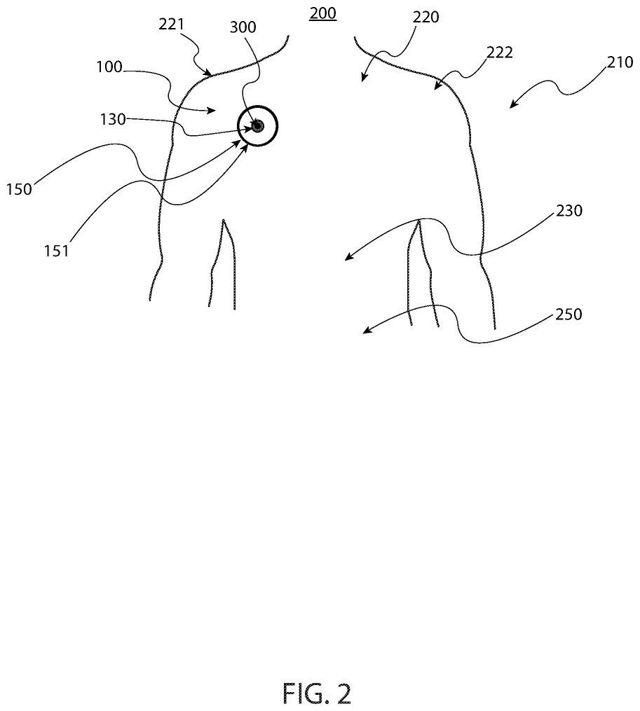

Moving now to FIG. 2, an illustrated view of a use of the port cap shown in FIG. 1A is presented.

A patient 200 has a torso 210. The torso 210 has an upper portion 220, a middle portion 230 and a bottom portion 250. The upper portion 220 of the torso 210 of the patient 200 has a right shoulder 221 and a left shoulder 222.

A port 300 for access to the blood stream is coupled to the blood stream through the torso 210. The port 300 is configured to be, for this example, in the upper portion 220 significantly near the right shoulder 221 of the patient 200.

The port cap 100 is configured such that the middle portion 130 of the port cap 100 is centered over the port 300. The bottom 150 is configured to couple to the torso 210 by the adhesive 151, see FIG. 1B. The port cap 100 is preferably applied to the port 300 at least one (1) hour prior to chemotherapy or blood draw.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A port cap for application to a port allowing access to a blood stream, the port cap consisting of:
a top;
a middle portion, the middle portion configured to be a reservoir, wherein the middle portion for insertion of a numbing agent, wherein the port cap having a circular shape; and
a bottom portion, the bottom portion having an adhesive, wherein the adhesive for being securely and removably coupled over a port of a patient's skin.

2. (canceled)

3. The port cap of claim 1, wherein the port cap being made of a flexible material.

4. The port cap of claim 3, wherein the flexible material being latex.

5. The port cap of claim 1, wherein the port cap being clear in color.

6. The port cap of claim 1, wherein the adhesive being a compound material.

7. The port cap of claim 6, wherein the compound material being silicone.

8. The port cap of claim 1, wherein the numbing agent being lidocaine.

9. The port cap of claim 1, wherein the numbing agent being distributed through the bottom portion of the port cap.

10. The port cap of claim 1, wherein the port cap being coupled to the patient for at least one (1) hour prior to chemotherapy or a blood draw.

11. A port cap for application to a port allowing access to a blood stream, the port cap consisting of:
a top;
a middle portion, the middle portion configured to be a reservoir, wherein the middle portion for insertion of a numbing agent, wherein the port cap having a circular shape, wherein the numbing agent being lidocaine, and wherein the numbing agent being distributed through the bottom portion of the port cap; and
a bottom portion, the bottom portion having an adhesive, wherein the adhesive for being securely and removably coupled over a port of a patient's skin, wherein the port cap being made of a flexible material, wherein the flexible material being latex, wherein the port cap being clear in color, wherein the adhesive being a compound material, wherein the compound material being silicone, and wherein the port cap being coupled to the patient for at least one (1) hour prior to chemotherapy or a blood draw.
